(19) United States
(12) Patent Application Publication
(10) Pub. No.: US 2015/0265157 A1
(43) Pub. Date: Sep. 24, 2015

(54) METHOD AND APPARATUS FOR A PHOTOACOUSTIC PROBE USING A MULTIMODE FIBER

(71) Applicant: ECOLE POLYTECHNIQUE FEDERALE DE LAUSANNE (EPFL), Lausanne (CH)

(72) Inventors: Demetri PSALTIS, Preverenges (CH); Christophe MOSER, Lausanne (CH)

(73) Assignee: ECOLE POLYTECHNIQUE FEDERALE DE LAUSANNE (EPFL), Lausanne (CH)

(21) Appl. No.: 14/665,113

(22) Filed: Mar. 23, 2015

(30) Foreign Application Priority Data
Mar. 24, 2014 (IB) PCT/IB2014/060094

(51) Int. Cl. A61B 5/00; A61B 1/00; A61B 1/07; G02B 6/036; G01H 9/00

(52) U.S. Cl. CPC A61B 5/0095; G02B 6/036; G01H 9/004; A61B 1/07; A61B 1/00009

(57) ABSTRACT

This invention is a method and apparatus to pick up a photoacoustic signal purely via optical means and via an endoscope. The photoacoustic endoscope invention relies only on optical excitation and detection methods. A light focus spot generates a photoacoustic signal at the distal end of a multimode fiber endoscope. A membrane is positioned at the distal end whose vibration is excited by the acoustic signal. A light beam brought at the distal end of the multimode endoscope via the single mode core of a dual clad multimode fiber is reflected by the said vibrating membrane. The light reflected is capture by the multimode core of the dual clad fiber and propagates to the proximal end. The speckle pattern thus generated at the proximal end depends on the membrane acoustic vibration. By correlating the speckle pattern with a known reference pattern stored prior to the generation of the photoacoustic signal, the signal can be sent to a single detector for the purpose of collecting more power and for detecting the fast acoustic signal which is in the range of Mhz to several tens of MHz.

### FIELD OF THE INVENTION

The invention is in the field of photoacoustic endoscopy.

### BACKGROUND

Photoacoustic endoscopy is an evolving imaging technique that is capable of delivering multi-scale images based on the optical absorption properties inside the body.

Recently, different groups have explored the possibility of building functional ultra-thin imaging devices based solely on multimode fibers exploiting the large number of degrees of freedom available in these waveguides. Fluorescent and linear scattering imaging has been demonstrated [1-3].

The present inventors have demonstrated an optical-resolution photoacoustic imaging modality in which a multi-mode fiber is used as the source of the optical excitation field. The present inventors used Digital Phase Conjugation (DPC) to focus and scan a diffraction-limited spot at the distal tip of the multimode fiber. This passive endoscope doesn't need optical lenses or mechanical actuators [4] leading to a very thin device the diameter of which is only limited by the fiber diameter. Furthermore, within a single approach a multimode fiber system could deliver multi-modal images based on fluorescence, linear scattering, and optical absorption through the photoacoustic effect.

It is an aim of the present invention, to provide a new concept of an ultrathin optical resolution photoacoustic endoscope that is based on a single multimode fiber. The scanning of the focus spot is achieved through a lensless and digital mechanism and the photoacoustic signal is picked up through the same fiber. In this way, both excitation and detection are based on optical mechanisms.

### SUMMARY OF THE INVENTION

In a first aspect the invention provides an apparatus for a photoacoustic probe comprising a double-clad fiber, a distal end of which is intended to be in contact with the photoacoustic probe to measure and equipped with an acoustic sensitive surface, whereby the double-clad fiber comprises a single mode core and a multimode cladding; the double-clad fiber being configured to guide with the single mode core a probe beam from a proximal end opposite to the distal end, to the distal end; whereby the acoustic sensitive surface is further arranged to reflect the probe beam, which thereafter is intended to reach the multimode cladding in order to excite the multimode cladding and generate a speckle pattern at the procimal end; further wherein the acoustic sensitive surface comprises a membrane configured to vibrate with sound along a preferential direction, and further coated such to reflect light coming from the probe beam.

In a second aspect the invention provides a method for a photoacoustic probe using a multimode fiber endoscope, comprising guiding a probe beam through a single mode core of the multimode fiber endoscope from a proximal end to a distal end, which is intended to be in contact with the photoacoustic probe to measure; positioning a membrane at the distal end; focusing the probe beam in a light focus spot on the photoacoustic probe, thereby generating a photoacoustic signal; exciting a vibration of the membrane by the photoacoustic signal; reflecting the probe beam at the distal end back by means of the membrane onto a multimode cladding of the multimode fiber endoscope; propagating the reflected probe beam towards the proximal end; measuring a speckles pattern at the proximal end as a function of the vibration of the membrane; and analyzing the speckle pattern.

In a preferred embodiment the step of focusing comprises scanning and focusing the light focus spot with Digital Phase Conjugation (DPC).

In a further preferred embodiment the step of analyzing the speckle pattern comprises correlating the speckles pattern with a known reference pattern.

In a further preferred embodiment the step of analyzing comprises determining an absorption contrast as a function of an amplitude of the the vibration of the membrane.

### DESCRIPTION OF THE DRAWINGS

The invention will be better understood through the detailed description of example embodiments and in reference to the drawings, wherein

FIG. 1 illustrates and example of a photo-acousto-optic probe according to an example embodiment of the invention;

FIG. 2 shows an overall design of the system for the detection of the photo-acousto-optic signal according to an embodiment of the invention;

FIG. 3 contains a reference speckle pattern at the proximal end of a fiber according to the invention; and

FIG. 4 contains a map of the cross-correlation coefficient between patterns generated by scanning the excitation and a reference pattern.

### DETAILED DESCRIPTION

The photoacoustic endoscope concept proposed here relies only on optical excitation and detection methods. A light focus spot generates a photoacoustic signal that is converted again to an optical signal and detected through optical means.

This invention is a method and apparatus to pick up a photoacoustic signal purely via optical means and via an endoscope. The photoacoustic endoscope invention relies only on optical excitation and detection methods. A light focus spot generates a photoacoustic signal at the distal end of a multimode fiber endoscope. A membrane is positioned at the distal end whose vibration is excited by the acoustic signal. A light beam brought at the distal end of the multimode endoscope via the single mode core of a dual clad multimode fiber is reflected by the said vibrating membrane. The light reflected is capture by the multimode core of the dual clad fiber and propagates to the proximal end. The speckle pattern thus generated at the proximal end depends on the membrane acoustic vibration. By correlating the speckle pattern with an known reference pattern stored prior to the generation of the photoacoustic signal, the signal may be sent to a single detector for the purpose of collecting more power and for detecting the fast acoustic signal which is in the range of Mhz to several tens of MHz.

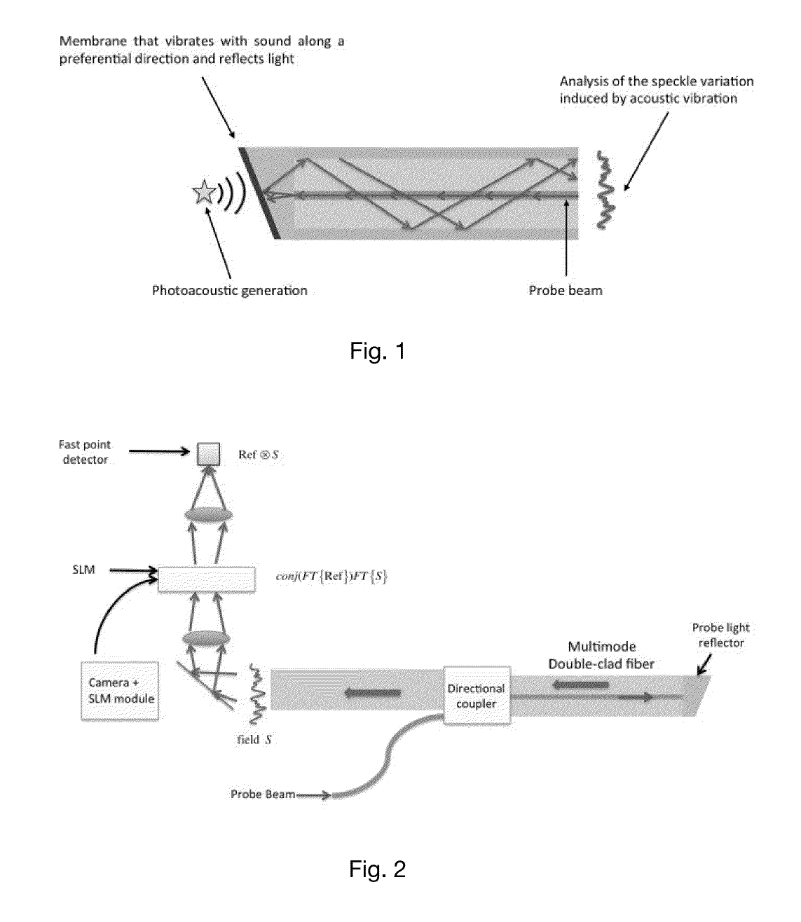

Looking at FIG. 1, we propose to use a double-clad fiber—a fiber with a single mode core and multimode cladding—to generate and scan a focus spot thanks to Digital Phase Conjugation (DPC) [3]. The distal end of the fiber—the side that is in contact with the sample—is designed in a way where it is equipped with an acoustic sensitive surface. The surface may for example be realized using a membrane—see FIG. 1. The membrane is coated in order to reflect light coming from a probe beam—generated from the single-mode core of the double-clad fiber. After reflection on the membrane, the probe beam excites the multimode cladding at the distal which generates a speckle pattern at the proximal end, i.e., detection side.

Once the photoacoustic signal is generated, an acoustic wave propagates to the membrane, which vibrates. The excitation of the multimode cladding by the probe beam will depend on these vibrations.

Since the mode distribution of a multimode fiber is very sensitive to the excitation condition, the speckle pattern at the proximal end will also change with the generated vibrations. By analyzing these changes, the absorption contrast could be linked to the amplitude of the vibrations, i.e to the speckle decorrelation coefficient. The speckle decorrelation speed would be directly linked to the generated acoustic wave frequency. Therefore by quantifying the speckle correlations to a reference speckle pattern, we could quantify the amplitude of the vibrations and thus the photoacoustic signal. A similar process has been demonstrated to compensate for bending while focusing through a multimode fiber by digital phase conjugation [5].

Below are listed the different steps proposed for the photoacousto-optic probe:
1. Generation and scanning of a pulsed focus spot by Digital Phase Conjugation through a multimode fiber.
2. The light pulse induces a photoacoustic effect generating an acoustic wave.
3. Conversion of the acoustic signal to an optical signal to be detected through the same fiber:
   a. The acoustic wave generated through the photoacoustic effect induces vibrations of a membrane.
   b. The membrane reflects a light probe beam of light coming from the single mode core of the fiber.
   c. The reflection of this probe beam will excite the fiber at its distal end.
   d. The speckle field at the proximal end is analyzed and the changes will be related to the amplitude and frequency of the membrane vibration, i.e to the photoacoustic signal.

### Measurement of Speckle Correlations:

The speckle field at the proximal end generated through by the membrane vibration is compared to a reference speckle field. We measure the cross-correlation of these two fields and this measurement has to be resolved in time to follow the photoacoustic signal. A way to do it is to use a fast detector that measures directly this cross-correlation coefficient. For this purpose, the generated speckle field at the proximal end is directed to a spatial light modulator that project the phase of the Fourier transform of the reference speckle field. The spatial light modulator is placed at the Fourier plane of a lens. The diffraction of the generated speckle field on the SLM, is then focused on a single detector placed at the conjugate plane of the SLM. This detector will then measure directly the cross-correlation coefficient between the generated speckle field and the reference speckle field (FIG. 2).

### Preliminary Results: Speckle Correlations as a Function of the Fiber Excitation:

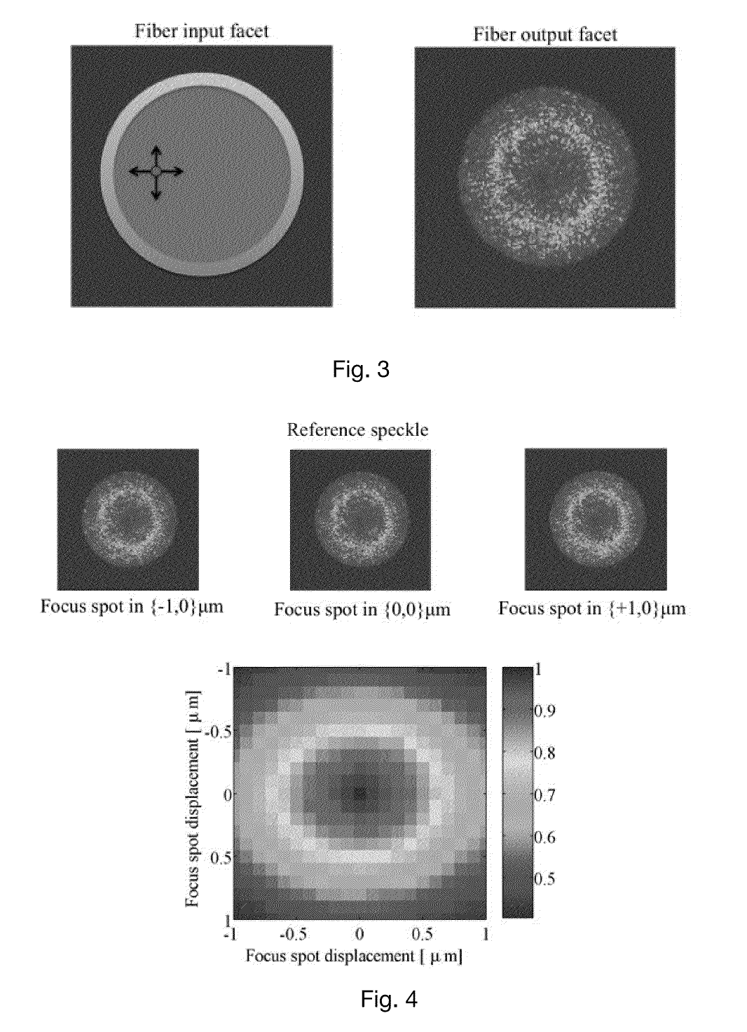

We experimentally measured the cross-correlation coefficient between a reference speckle pattern and speckle patterns generated by scanning a focus spot. As shown in FIG. 3, a laser beam focusing at the input facet of a MMF generates a speckle pattern at the output facet.

Scanning the focus spot within a small region around the reference position, a degree of correlation between the generated speckle patterns and the first output has been observed.

In particular, the focus spot is scanned over a square of 2 μm side with steps of 100 nm. The speckle generated at the center of the scanned grid is considered as reference.

The calculated cross correlation coefficient shown in FIG. 4 reveals a 60% of similarity between the patterns generated using focus spots 1 μm far apart from each other.

### REFERENCES

1. S. Bianchi and R. Di Leonardo, “A multi-mode fiber probe for holographic micromanipulation and microscopy.,” Lab on a chip 12, 635-9 (2012).
2. T. \check{C}i\check{z}már and K. Dholakia, “Exploiting multimode waveguides for pure fibre-based imaging,” Nature Communications 3, 1027 (2012).
3. I. Papadopoulos and S. Farahi, “High-resolution, lensless endoscope based on digital scanning through a multimode optical fiber,” Biomedical Optics Express 4, 17598-17603 (2013).
4. I. N. Papadopoulos, 0. Simandoux, S. Farahi, J. Pierre Huignard, E. Bossy, D. Psaltis, and C. Moser, “Optical-resolution photoacoustic microscopy by use of a multimode fiber,” Applied Physics Letters 102, 211106 (2013).
5. S. Farahi, D. Ziegler, and I. Papadopoulos, “Dynamic bending compensation while focusing through a multimode fiber,” Optics Express 21, 510-512 (2013).

1. An apparatus for a photoacoustic probe comprising:
a double-clad fiber, a distal end of which is intended to be in contact with the photoacoustic probe to measure, and equipped with an acoustic sensitive surface, whereby the double-clad fiber comprises a single mode core and a multimode cladding;
the double-clad fiber being configured to guide with the single mode core a probe beam from a proximal end opposite to the distal end, to the distal end;
whereby the acoustic sensitive surface is further arranged to reflect the probe beam, which thereafter is intended to reach the multimode cladding in order to excite the multimode cladding and generate a speckle pattern at the proximal end;
further wherein the acoustic sensitive surface comprises a membrane configured to vibrate with sound along a preferential direction, and further coated such to reflect light coming from the probe beam.

2. A method for a photoacoustic probe using a multimode fiber endoscope, comprising
guiding a probe beam through a single mode core of the multimode fiber endoscope from a proximal end to a distal end, which is intended to be in contact with the photoacoustic probe to measure;
positioning a membrane at the distal end;
focusing the probe beam in a light focus spot on the photoacoustic probe, thereby generating a photoacoustic signal;
exciting a vibration of the membrane by the photoacoustic signal;
reflecting the probe beam at the distal end back by means of the membrane onto a multimode cladding of the multimode fiber endoscope;
propagating the reflected probe beam towards the proximal end;
measuring a speckles pattern at the proximal end as a function of the vibration of the membrane; and
analyzing the speckle pattern.

3. The method of claim 2, wherein the step of focusing comprises scanning and focusing the light focus spot with Digital Phase Conjugation (DPC).

4. The method of claim 2, wherein the step of analyzing the speckle pattern comprises correlating the speckles pattern with a known reference pattern.

5. The method of claim 2, wherein the step of analyzing comprises determining an absorption contrast as a function of an amplitude of the the vibration of the membrane.
